(19) United States
(12) Patent Application Publication
(10) Pub. No.: US 2009/0047280 A1
(43) Pub. Date: Feb. 19, 2009

(54) Antibody inhibiting infection of papillomavirus

(76) Inventors: Patricio I. Meneses, Vernon Hills, IL (US); Erle S. Robertson, Wannewood, PA (US); Kha Nguyen, Chicago, IL (US); Valerie Laniosz, North Chicago, IL (US)

Correspondence Address: Joseph A. Fuchs;Rockey, Depke & Lyons, LLC, 233 S. Wacker Drive, Suite 5450, Sears Tower, Chicago, IL, 60606-6306, US

(21) Appl. No.: 12/006,928

(22) Filed: Jan. 7, 2008

Related U.S. Application Data
(60) Provisional application No. 60/879,163, filed on Jan. 8, 2007.

(51) Int. Cl. A61K 39/395; C07K 16/18; C12N 5/06; A61P 31/00

(52) U.S. Cl. 424/133.1; 530/387.9; 530/387.3; 424/139.1; 435/375

(57) ABSTRACT

The present invention relates generally to an antibody that binds specifically to a neutralization epitope at the surface of papillomavirus (residues 36-49 of the minor capsid L2) that does not interfere with virus entry into endosomes and lysosomes. This antibody neutralizes infection of the virus in a dose dependent manner.

### CROSS-REFERENCE TO RELATED APPLICATION

The present application claims priority to and the benefit of U.S. Provisional Application No. 60/879,163 filed on Jan. 8, 2007, and is incorporated by reference and made a part hereof.

### FEDERALLY SPONSORED RESEARCH OR DEVELOPMENT

This invention was made with government support under National Institutes of Health (NIH)/National Cancer Institute (NCI) grant number K22:CA117971. The government has certain rights in the invention.

### TECHNICAL FIELD

The present invention relates generally to an antibody that binds specifically to a neutralization epitope at the surface of papillomavirus (residues 36-49 of the minor capsid L2) that does not interfere with virus entry into endosomes and lysosomes. This antibody neutralizes infection of the virus in a dose dependent manner.

### BACKGROUND OF THE INVENTION

Papillomavirus (PV) infection is the most common sexually transmitted disease in the U.S. (approximately 5.5 million per annum) (40). PVs induce a variety of lesions such as cutaneous and genital warts. Specific human PVs (HPVs, high-risk) are the etiologic agent of cervical carcinoma, a major killer of women worldwide (26, 48-50). The number of cervical cancer occurrences in the U.S. has been reduced by 70% with the development of Dr. Papanicolau's screening known as the “Pap smear”. The Pap smear test is readily available in the US and other western countries, but is not effectively used in underdeveloped countries. There are 500,000 cervical cancer cases diagnosed worldwide and 288,000 deaths from the disease yearly (45). Cervical cancer is the second leading cause of death for women worldwide with a median age of 38 and 90-95% of these cancers are positive for specific HPV DNA (18, 28).

PVs are small double stranded circular DNA viruses that infect a wide range of organisms (38, 64). Genomic analysis of the various PVs shows a high degree of similarity (14). The PV genomes typically contain 10 open reading frames (ORFs), which are all expressed from one strand. These ORFs are divided into two classes: early genes (E1-E8) and late genes (L1 and L2) (38). All PVs have a non-coding region referred to as the long control region (LCR) that contains the viral origin of replication as well as transcription regulatory elements (38). The life cycle of PV parallels the differentiation of the epithelium. In the basal cells of the epithelium where primary infection occurs, a low copy number of viral genomes is maintained. While uninfected basal cells normally exit the cell cycle during differentiation, PV infected cells continue with active cell division despite ongoing differentiation. This provides the cellular machineries necessary for viral genome replication and for the production of PV capsid proteins L1 and L2. As cells differentiate, the level of viral genomes increases and mature virions are formed in the upper layers.

The entry of PVs into cells has been reported to depend on receptor mediated endocytosis. BPV-1, HPV-16 and HPV 58 have been shown to enter via clathrin mediated vesicles, and to co-localize with endosome markers such as eeA1 and the lysosome marker LAMP1 (5, 16, 57, 62). Although HPV-31 pseudovirions, or viral like particles (VLPs), generated only with L1 protein have been shown to internalize by caveolae in-vitro (5), most studies indicate the lack of inhibition of infection using caveolae inhibitors. In contrast, blocking clathrin/endosome mediated entry results in the loss of infection. These studies suggest that the entry of PVs, and thus viral tropism, may depend on the viral capsid components as well as cellular receptor availability, in particular receptors leading to entry via endosomes. One theme that emerges in the PV entry studies is the necessity of both the L1 and L2 viral capsid proteins for efficient infection.

PV capsids consist of L1 and L2 at an estimated ratio of 30:1 (20, 60). The L1/L2 ratio suggests that there is one L2 at each of 12 capsid vertices (60). L2 is referred to as the minor viral capsid protein in this non-enveloped, icosahedral virion (1). The virus is assembled in the nucleus of squamous epithelial cells into particles 52-55 nm in diameter (11). Each viral particle contains a single double stranded circular DNA genome bound by histone proteins (19, 27). Expression of viral L1 protein in the absence of other viral proteins results in the packaging of DNA at inefficient levels (7, 61). The addition of L2 in the viral particle production increases DNA packaging into virions resulting in increased infectivity (31, 63), and L2 contributes to the entry of the virions into cells (4, 22). Antibodies to L2 have proved to be neutralizing and have led to the finding that a portion of L2 is exposed on the capsid surface (10, 25, 35, 41, 54). Several studies have addressed the use of papillomavirus L2 sequences as vaccine targets since these regions of L2 shown to be exposed on the capsid surface are more conserved than the loops of the L1 protein (35, 39, 46, 55). The approaches in these studies have demonstrated that a linear epitope at residues 108-120 can be targeted by a monoclonal antibody, and that various anti-sera to peptides derived from the n-terminus of L2 are neutralizing across genotypes. Recent improvements in the process to generate virion titres of papillomavirus have made it possible to study the process of viral entry in detail (6, 51). These pseudovirions are capable of encapsidating plasmid DNA containing transgenes and full papillomavirus genotype genomes. These in-vitro generated pseudovirions have been used to study viral infection and neutralization, dendritic cell activation, and in studies dealing with the role of nuclear compartments, known as ND10s, and cellular proteins such as dynein and syntaxin 18 (4, 15, 23, 24, 39). In addition, pseudovirions generated in this fashion containing the cottontail rabbit papillomavirus (CRPV) genome were recently shown to be infectious in-vivo, i.e., papillomatous growths were induced (12). These studies show that the current strategy of pseudovirion production in 293TT cells results in proper viral particle formation, with biology analogous to genuine virus.

We previously used a proteomics approach to identify L2 interacting cellular proteins (4). Our data showed the interaction of L2 with a cellular protein, syntaxin 18, involved in intracellular vesicle trafficking. We identified that the region of L2 encompassing residues 41-44 was important for infection since mutation of these residues resulted in loss of infection. Mutation of these residues also led to a loss of co-immunoprecipitation of BPV-1 L2 protein with syntaxin 18 protein. Our results suggested that residues 41-44 were exposed on the outer surface of the virion, and played a role in trafficking of the virions and in mediating the interaction of L2 with syntaxin 18. In the present disclosure, we used an affinity purified antibody to BPV-1 L2 residues 36-49 to demonstrate that this region of L2 is indeed on the surface of the viral particles. We observed neutralization of infection with this antibody, but show no block of the entry of the virions into early endosomes or lysosomes. This antibody does interfere with the co-immunoprecipitation of L2 with syntaxin 18.

Focusing on the role played by L2 during infection may lead to a better understanding of how papillomaviruses manipulate and use the cellular machinery to allow for viral infection.

These and other aspects and attributes of the present invention will be discussed with reference to the following drawings and accompanying specification.

### SUMMARY OF THE INVENTION

In an embodiment, the present invention discloses an isolated antibody, or antigen-binding fragment thereof, that binds specifically to residues 36-49 of a papillomavirus (PV) minor capsid L2. The antibody or the antigen-binding fragment thereof can be used to inhibit infection of a mammalian cell by the papillomaviruses (PVs). In an embodiment, the PV is bovine papillomavirus. In another embodiment, the PV is bovine papillomavirus 1 (BPV-1) and wherein the residues 36-49 have a peptide sequence of SEQ ID NO:1. In yet another embodiment, the PV is human papillomavirus (HPV). The antibody can be polyclonal or monoclonal. In a preferred embodiment, the antibody is humanized.

In a further embodiment, the present invention discloses a composition for inhibiting infection of a mammalian cell by a papillomavirus, the composition comprises an isolated antibody, or antigen-binding fragment thereof, that binds specifically to residues 36-49 of the papillomavirus (PV) minor capsid L2. In an embodiment, the PV is bovine papillomavirus. In another embodiment, the PV is bovine papillomavirus 1 (BPV-1) and wherein the residues 36-49 have a peptide sequence of SEQ ID NO:1. In yet another embodiment, the PV is human papillomavirus (HPV). The antibody can be polyclonal or monoclonal. In a preferred embodiment, the antibody is humanized. Preferably, the composition has a pharmaceutically acceptable carrier. The antibody can polyclonal or monoclonal. Preferably, the antibody is humanized.

In a still further embodiment, the present invention discloses a method for inhibiting infection of a mammalian cell by a papillomavirus, the method comprises providing the mammalian cell with an effective amount of a composition comprising a molecule that binds specifically to residues 36-49 of the papillomaviruses (PV) minor capsid L2. In an embodiment, the PV is bovine papillomavirus (BPV). In another embodiment, the PV is bovine papillomavirus-1 (BPV-1) and the residues 36-49 have a peptide sequence of SEQ ID NO:1. In yet another embodiment, the PV is human papillomavirus (HPV). The molecule can be a macromolecule or a small molecule. In a preferred embodiment, the macromolecule is an antibody, or an antigen-binding fragment thereof. The antibody can be polyclonal or monoclonal. Preferably, the antibody is humanized.

In yet another embodiment, the present invention discloses a composition for generating an antibody that binds specifically to residues 36-49 of a papillomaviruses (PV) minor capsid L2, the composition comprising a peptide comprising residues 36-49 of a papillomaviruses (PV) minor capsid L2. In a preferred embodiment, the peptide is conjugated to keyhole-limpet hemocyannine (KLH) at the N-terminal via a cysteine moeity.

In yet another further embodiment, the present invention discloses an isolated antibody, or antigen-binding fragment thereof, that binds specifically to residues 36-49 (SEQ ID NO:1) of bovine papillomavirus-1 (BPV-1) minor capsid L2. The antibody or the antigen-binding fragment is capable of inhibiting infection of a mammalian cell by bovine papillomavirus-1 (BPV-1) or human papillomavirus 16 (HPV-16).

### BRIEF DESCRIPTION OF THE DRAWINGS

FIG. 1 shows an antibody made against BPV-1 L2 residues 36-49 demonstrates epitope specificity.

FIG. 1A: BPV-1 L2 residues 36-49 with the addition of an N-terminal sequence were linked to KLH for antibody production.

FIG. 1B: Lane 1: Diagram of L2 indicating the DNA binding domains, and the L1 interacting domains. Diagram of BPV-1 L2 deleted of residues 41-54 (Lane 2), 31-44 (Lane, and 41-44 (Lane 4).

FIG. 1C: Western blot of Cos-7 cell lysates transfected with: BPV-1 Full length (Lane 1), deleted of residues 41-54 (Lane 2), deleted of residues 31-44 (Lane 3), deleted of residues 41-44 (Lane 4). Upper panel was blotted with anti-Full length L2 antibody; middle panel was blotted with anti-L2 residues 36-49 antibody; and bottom panel was blotted with pre-immune sera;

FIG. 2 shows the result of electron microscopic analysis of binding to BPV-1 pseudovirions by antibody against residues 36-49. BPV-1 pseudovirions generated in 293TT cells purified over optiprep layers were pre-incubated with either affinity purified antibody made against BPV-1 L2 residues 36-49 (A,B,C) or with affinity purified anti GST antibody (D,E,F). Scale bar represents 100 nm at 120,000× magnification. Black dots are the 10 nm gold labeled anti rabbit secondary antibody, the virus is approximately 50 nm in diameter;

FIG. 3 shows the affinity purified antibody corresponding to L2 residues 36-49 exhibits a dose dependent neutralization of infection when pre-incubated with BPV-1 pseudovirions. FACS analysis of the percent of Cos-7 cells infected with pseudovirus alone (A and B, gray bars) is compared to Cos-7 cells infected with the virus that was pre-incubated with the affinity purified antibody made against L2 36-49 in B, and when the virus was pre-incubated with the affinity purified isotypic anti-GST antibody control antibody in A. Antibody concentrations used were 2, 10, and 20 ug. Each bar represents the average of three infections and the error bars show the standard deviation. Experiment was performed a minimum of 3 times with similar results;

FIG. 4 shows the neutralization by the antibody made against BPV-1 L2 36-49 can be reversed by pre-incubating the antibody with a peptide corresponding to residues 36-49. Infections with pseudovirions carrying the GFP cDNA plasmid 8fwb were performed in the presence of antibody against residues 36-49 that was pre-incubated with BPV-1 L2 peptides in order to address if infection block was antibody specific. The affinity purified antibody generated against BPV-1 L2 residues 36-49 was pre-incubated with increasing amounts of the peptide corresponding to residues 36-49 from 1-100 uM (A) and 10 nM-1 uM (B), or was pre-incubated with a control peptide, made with the BPV-1 L2 residues 36-49 in random order, at 1-100 uM, (C) and 10 nM-1 uM (D). Infection percentage was determined by FACS analysis of GFP positive cells. Each bar represents the average of three trials in which 10,000 events were counted. The standard deviation is demonstrated by the error bars at the top of each column;

FIG. 5 shows BPV-1 pseudovirions enter EEA1-positive compartments even in the presence of the neutralizing antibody made against L2 residues 36-49. Analysis of pseudovirus entry was analyzed in the presence of the neutralizing antibody. Cos-7 cells were infected with BPV-1 in the absence (A, B) and presence (C-H) of the affinity purified antibody made against L2 amino acids 36-49. 5B6, seen as red in A-F, was used to stain L1 of the pseudovirion. EEA1 corresponds to an early endosomes marker and is seen in A, B, E, and F as green and is stained red in G and H. The antibody made against L2 residues 36-49 is stained green in C, D, G, and H. TOPRO-3 staining was used to visualize the nucleus. Z-stacked images, which display a three dimensional view of the images (B, D, F, and H) were taken from the merged image. Co-localization of red and green labeling markers is seen as yellow. The region of the image in A, C, E, and G that the Z-stack was taken of is indicated by the grey arrows;

FIG. 6 shows BPV-1 pseudovirions are found in LAMP-1 positive compartments in the presence of the neutralizing antibody against L2 residues 36-49 at 2 and 4 hours post-infection. Trafficking of the virions was analyzed in the presence of the neutralizing antibody at 2 and 4 hours. Z-section, (XYZ) plane images are shown as merge images in B, D, F, H, J, L, N, P, R, and T. Cos-7 cells were infected with BPV-1 pseudovirions for 2 hours (A-J) or 4 hours (K-T). Cells in A, B, K, and L were infected with pseudovirions and were stained with LAMP-1 and the conformation dependent anti-L1 antibody 5B6. Cells in C, D, M, and N were infected with pseudovirions that was pre-incubated with neutralizing antibody (and thus antibody was bound to the pseudovirions) made against L2 residues 36-49 and stained with LAMP-1 and 5B6 (C and D) or LAMP-1 and conformation independent anti-L1 antibody 1H8 (M and N). Cells in E, F, O, and P were infected with pseudovirions bound by the neutralizing antibody against residues 3649 and were stained for the bound antibody using an anti-rabbit labeled antibody (α-rabbit) and 5B6. Cells in G, H, Q and R were infected with pseudovirions bound to neutralizing antibody and stained for bound antibody and LAMP-1. Cells infected with pseudovirus bound by neutralizing antibody were stained with anti-LAMP-1, anti-L1 5B6, and anti-rabbit antibodies (I and J), or with anti-L1 1H8, anti-LAMP-1, and anti-rabbit antibodies (S and T). TOPRO-3 was labeled with alexa-fluor 642, and we used donkey anti-rabbit and anti-mouse alexa-fluor 488, donkey anti-rabbit and anti-mouse 594, donkey anti-rabbit 642, and chicken anti-goat (for goat LAMP1 on I, J, and T) 594 as secondary antibodies. Primary antibodies were mouse anti-L1 5B6, mouse anti L1 1H8, rabbit anti LAMP-1 in A-D and K-N, and goat anti LAMP-1 in G-J, and Q-T. Confocal images were acquired on an Olympus Fluoview 300 under 60× oil and 2.5× magnification. Merge image enlargements from the acquired Fluoview 300 images were digitally enhanced 5× in the Z sections. Overlap of signals is seen as yellow staining in A-H and K-R, or in white in I, J, S, and T;

FIG. 7 shows the antibody competes away the interaction of L2 with syntaxin 18. The ability of the antibody to compete for binding of syntaxin 18 and BPV-1 L2 was tested by antibody co-immunoprecipitation. In panels A and B, cells were transfected with control vector pA3M (lane 1), pA3M and BPV-1 L2 (lane 2), pA3M and Flag-syntaxin 18 (lane 3), or BPV1 L2 and Flag-syntaxin 18 (lane 4). Samples were immunoprecipitated with the affinity purified antibody against residues 36-49 (A), or anti-Flag M2 antibody (B). Western blots for L2 (top) and Flag-syntaxin 18 (bottom) are shown;

FIG. 8 shows a computer generated BPV-1 L2 structure. Purple sequence encompassing residues 1-88 is shown on the model as a separate domain from the rest of BPV-1 L2. In blue is the aspartic acid at position 40 and in yellow are residues 41-KILK-44. The residues DKILK are in an alpha helix domain. In red is the methionine at position 1;

FIG. 9 is a comparison of residues 36-49 of BPV-1 L2 with other PV genotypes. Comparison of residues corresponding to position 36-49 of BPV-1 L2 (residues 40-44 shown in bold). BPV1, 2-Bovine Papillomavirus; HPV 35, 11, 16, 33, 31-Human Papillomavirus; COPV, Canine Oral Papillomavirus; FdPV, Felis-domesticus Papillomavirus.; and

FIG. 10 shows HPV 16 pseudovirion infection is neutralized with the affinity purified antibody made against residues 36-49. 293TT were infected with 1 or 2 ul of HPV 16 pseudovirions, approximately 7×10⁴ infectious units/ul. Addition of 100 ug of affinity purified antibody against BPV-1 L2 inhibits infection by 35%.

FIG. 11 shows a coomassie gel checking the purity of the IgG purification. The final bleed was incubated with agarose beads coated with Protein A and G in order to bind any IgG. Beads were placed in a gravity drip column and fractions were collected using low pH buffer. Fractions 1-4 show a predominant band at the size of the heavy chain corresponding to IgG. The column flowthru was collected and run on the gel undiluted, and after 1:10 and 1:2 dilution in PBS. There is a band corresponding to IgG in the flowthru suggesting that some IgG was not bound although the majority bound and eluted in fractions 1-4.

FIG. 12 shows a Western blot detecting HPV16 L2 protein. Lysates from COS-7 cells transfected with HPV16L2 protein tagged with the HA epitope were run on an 8% SDS-PAGE and transferred onto nitrocellulose. Parallel western blot analysis was performed with total immune sera and anti-HA antibody (FIG. 2 A), or affinity purified (using the 16 L2 peptide, Frac 30) immune antibody provided by 4ADI and anti-HA antibody (FIG. 2 B). Control plasmid A3M (a derivative of pcDNA3) was transfected on control cells. Analysis of western blot using a Li-Cor Odyssey (Li-Cor, Lincoln, Nebr.) shows the Yellow overlap (arrow) of the L2 staining (Green) with the anti-HA staining (Red). This blot demonstrates that the affinity purified antibody in Frac 30 contains the IgG recognizing HPV16L2.

FIG. 13 shows HPV16 infection of COS-7 cells in the presence of the HPV16 L2 antibody 36-49 results in a loss of infection. COS-7 cells were infected HPV16 pseudovirions alone (HPV alone, 15%), the level of infection was reduced to 10% with 2 ug immune IgG, and further decrease in infection to 5% and 3% was observed with 10 ug and 20 ug of IgG respectively. Non-immunized normal rabbit sera (NRS) IgG was used as control. These data demonstrate a specific dose dependent block of neutralization.

### DETAILED DESCRIPTION OF THE INVENTION

While this invention is susceptible of embodiments in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

Papillomavirus (PV) has been identified as the etiologic agent responsible for cervical carcinoma. Efforts are ongoing trying to understand the biology of PV infection in order to develop a vaccine that would prevent infection and thus lower the level of PV malignancy worldwide, particularly cervical cancer.

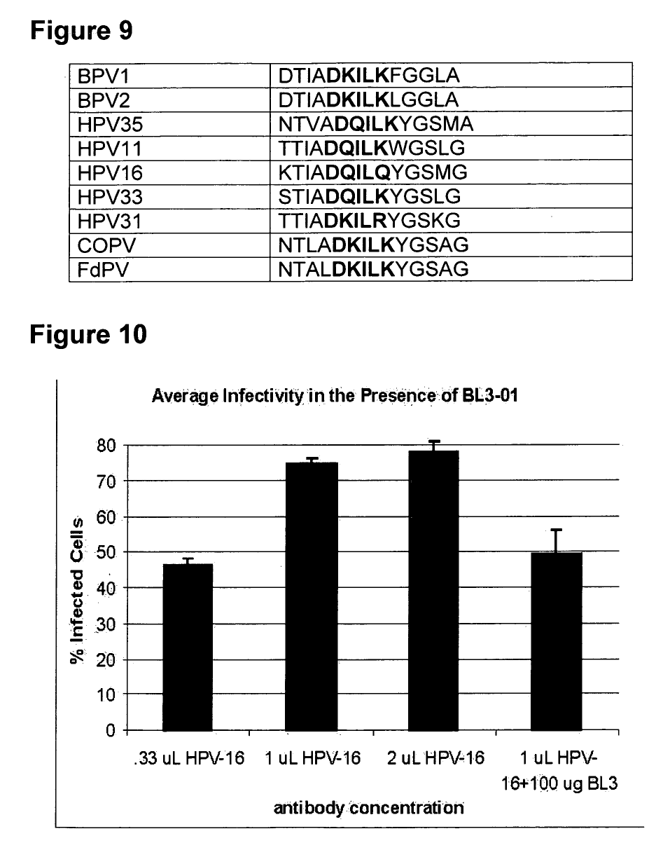

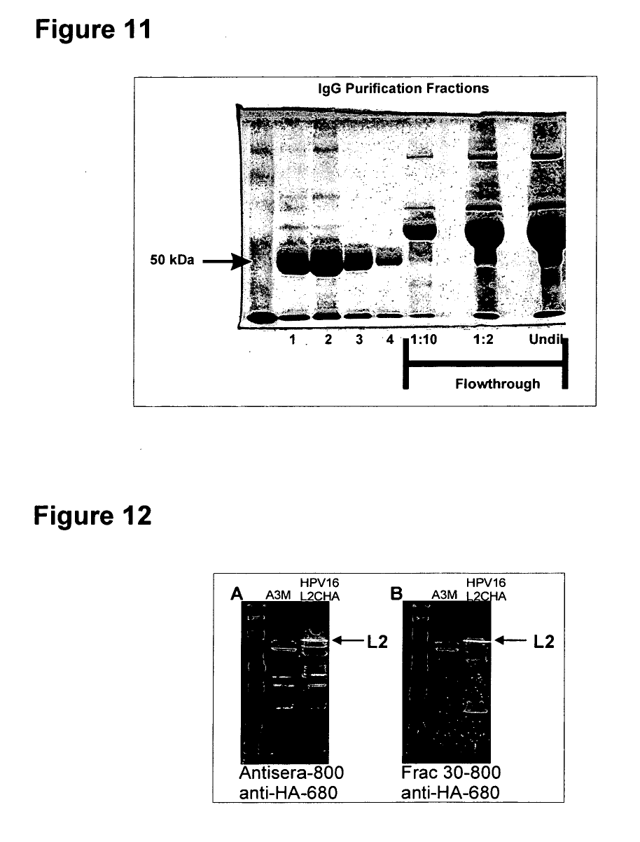

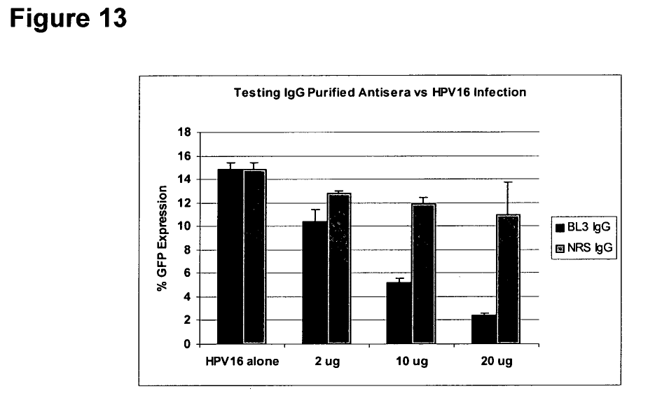

The present invention discloses a region of the minor capsid L2 of papillomaviruses, residues 36-49, that is involved in infection by mediating viral entry. This region of L2 is exposed on the surface of the virions, and is highly conserved in the L2 proteins of papillomaviruses. This region is a neutralization epitope at the surface of papillomavirus that does not interfere with virus entry into endosomes and lysosomes. Comparison of amino acid residues 36-49 of L2 minor capsid protein of various papillomavirus genotypes are shown in FIG. 9. There is high conservation in these 15 residues, particularly residues 40-44 (FIG. 9 Bold DK/QILK/Q). The homology in this region is observed between genotypes of the same species and across species of papillomaviruses.

Understanding the mechanism of viral infection of papillomaviruses (PVs) is both clinically important, and biologically interesting. There are 13 HPV genotypes designated as oncogenic out of over 100 genotypes (32). Of these, HPV 16 and 18 are linked to approximately 70% of the identified cervical carcinoma cases (3). Other HPVs are medically important since they cause sexually transmitted disease and warts (8). Additionally, animal PVs cause disease and distress in companion and farm animals, with financial and emotional effects to the owners (reviewed in (9)).

In the present disclosure, we used the PV prototype, BPV-1, to analyze the role the minor capsid protein L2, in particular residues 36-49, in the early stages of infection. Previous work in our laboratory demonstrated that this region of L2, in particular residues 40-44, is important for infection (4). We describe herein how an antibody generated against a peptide corresponding to residues 36-49 of BPV1 L2 recognizes an exposed epitope on the viral particles, and was able to neutralize infection by BPV-1 pseudovirions. Neutralization occurred without preventing the internalization of the viral particles into early endosomes or lysosomes, consistent with clathrin-dependent receptor mediated endocytosis.

Viruses are able to exploit the cells' replication, transcription, and translation machineries for their own need. They are also able to exploit the various endocytosis mechanisms (47, 59). In order to complete an infection, DNA viruses need to deposit their genome and the necessary viral proteins into the nucleus of the infected cell where the viral genome is replicated and new infectious viral particles are made. Endocytosis offers a method that: 1) allows the virus to bypass the crowded cytoplasm into the perinuclear region; 2) does not allow for any viral protein to remain in the plasma membrane, thus limiting the immune response; 3) takes the virus into vesicles whose pH or other factors may assist in the completion of the viral infection. The major methods of internalization include a) clathrin dependent, b) caveolae dependent, and c) mediated by lipid rafts, or other, non-clathrin/non-caveolae pathways. After internalization via the above-mentioned methods, the various endocytosis pathway(s) sort their cargo to the proper cellular organelle. Caveolae mediated entry transports its cargo to caveosomes for sorting into the endoplasmic reticulum, Golgi, and perhaps endosomes. Clathrin mediated endocytosis transports its cargo into early endosomes where it is sorted into late endosomes, Golgi, or recycled to the plasma membrane. And finally, lipid rafts and other unknown pathway transport their cargo to the endosomes and perhaps caveosomes.

PV infection begins with the binding of the virus to the surface of the cells. The α₆B₄ integrin complex has been suggested as a potential receptor for PVs (21, 42). This receptor is expressed in epithelial cells, mesenchymal cells, and neurons. However, PVs can infect cells lacking this integrin, suggesting that alternate receptor usage exists (58). In agreement with this notion, PV virions have been shown to directly bind heparin and surface glycosaminoglycans on human foreskin keratinocytes prior to internalization (33). A recent study demonstrates the internalization of BPV-1 via clathrin-coated vesicles (16). In the present disclosure, the viral particles are shown to be co-localized with a clathrin adaptor molecule, the transferrin receptor, as a marker of early endosomes and Lamp-2 as a marker of late endosomes and lysosomes. Following internalization into an endosome, a furin cleavage and a c-terminal sequence have been shown to be required for PV viral endosome escape (34, 52). Infection proceeds with the disassembly of the virus within 6 hours after infection, and is completed after the viral DNA is transported into the nucleus by a mechanism that may require L2's ability to bind DNA and the nuclear import receptors (2, 13, 16, 37). The kinetics of infection are such that internalization occurs with a t_{1/2} of 4 hours, and transcription of viral packaged DNA occurs after 12 hours (16).

The initial step of binding to the cell membrane by PV is attributed to the viral L1 protein, with no contribution of the L2 protein (43, 53). Studies have suggested that the L2 protein contributes to the internalization of the virus, to the nuclear translocation of the viral DNA, to the escape of the viral DNA from endosomes, and to the intracellular trafficking of the virions (4, 15, 34, 56). Lack of L2 in viral particles has been repeatedly shown to result in loss of infectivity (31, 36).

Our laboratory had previously shown that BPV-1 L2 interacted with the intracellular trafficking molecule syntaxin 18 (4, 29). Disruption of intracellular trafficking with the dominant negative syntaxin 18 resulted in loss of infection, loss of perinuclear trafficking of BPV-1 pseudovirions, and lack of interaction with syntaxin 18 in transfected cells (4). We showed that mutation of residues 40-44 of BPV-1 L2 resulted in loss of infection (4). We disclose herein that an antibody generated against the region encompassing these residues demonstrates that these residues are exposed on the outer surface of the viral capsids. The finding of these residues on the outside of the viral capsids suggested that an antibody to these residues would result in the neutralization of the viral particles, much like the loss of infection that resulted with the mutation of these residues observed in our previous manuscript (4). We indeed observed a loss of infection when virions were incubated with antibody prior to infection. This infection block was prevented or competed away by the peptide used to generate the antibody in a dose dependent manner and not by a control peptide consisting of the same but randomly distributed sequence, thus showing that the block was specifically due to the affinity purified antibody against residues 36-49.

It had been previously observed that neutralization of papillomavirus infection with antibodies to L2 was not solely due to the prevention of virus binding to the cellular membrane (53, 54). We addressed the mode of neutralization of the antibody to residues 36-49, and indeed we did not see a loss of virus binding to the surface of the cells. In addition, we show that virus that was pre-incubated with the antibody against 36-49, and thus neutralized, was still co-localized with the early endosome marker eeA1 and the late endosome/lysosome marker LAMP-1, even after 4 hours. This staining demonstrates that as first described, neutralization of viral infection by targeting papillomaviruses may occur at a step subsequent to binding (53, 54).

Our computer generated model (http://robetta.bakerlab.org/) of BPV-1 L2 shown in FIG. 8 suggests that residues 40-44 are part of an alpha helix that resides on an exposed portion of L2. In purple are the n-terminal 88 residues of L2, in yellow are residues 41-44, and the aspartic acid at position 40 is in blue. This model led us to propose that this region of L2 was interacting with a cellular protein and was thus critical in the binding of the virions at the surface of the cells or in the trafficking of the virus. In this manuscript we show that this region is not involved in binding of the virus to the surface of cells. Our data suggests that this region of L2 is also not important for the movement of the virus from the plasma membrane into an endosome or lysosome. Further experiments need to be done to determine if this region of L2 is important for the escape of virus from endosomes, or for the subsequent trafficking of the virus or L2 through the cytoplasm. Our original observation was that this region, in particular residues 40-44, of L2 was mediating the interaction with the intracellular trafficking protein Syntaxin 18. A recent manuscript describes how syntaxin 18 is involved in the process of phagocytosis and mediating the fusion of plasma membrane derived vesicles with intracellular organelles by associating with plasma membrane-localized syntaxins (30). In the present disclosure, we show that the antibody against residues 36-49 of BPV-1 L2 prevents its co-immunoprecipitation with syntaxin 18.

In an embodiment, the present invention discloses an isolated antibody, or antigen-binding fragment thereof, that binds specifically to residues 36-49 of a papillomavirus (PV) minor capsid L2. The antibody or the antigen-binding fragment of can be used to inhibit infection of a mammalian cell by the papillomaviruses (PVs). The PV can by any PV. In an embodiment, the PV is bovine papillomavirus. In another embodiment, the PV is bovine papillomavirus 1 (BPV-1) and wherein the residues 36-49 have a peptide sequence of SEQ ID NO:1. In yet another embodiment, the PV is human papillomavirus (HPV). The antibody can be polyclonal or monoclonal. In a preferred embodiment, the antibody is humanized.

In a further embodiment, the present invention discloses a composition for inhibiting infection of a mammalian cell by a papillomavirus, the composition comprises an isolated antibody, or antigen-binding fragment thereof, that binds specifically to residues 36-49 of the papillomavirus (PV) minor capsid L2. The PV can be any PV. In an embodiment, the PV is bovine papillomavirus. In another embodiment, the PV is bovine papillomavirus 1 (BPV-1) and wherein the residues 36-49 have a peptide sequence of SEQ ID NO:1. In yet another embodiment, the PV is human papillomavirus (HPV). The antibody can be polyclonal or monoclonal. In a preferred embodiment, the antibody is humanized. Preferably, the composition has a pharmaceutically acceptable carrier. The antibody can polyclonal or monoclonal. Preferably, the antibody is humanized.

In a still further embodiment, the present invention discloses a method for inhibiting infection of a mammalian cell by a papillomavirus, the method comprises providing the mammalian cell with an effective amount of a composition comprising a molecule that binds specifically to residues 36-49 of the papillomaviruses (PV) minor capsid L2. The PV can be any PV. In an embodiment, the PV is bovine papillomavirus (BPV). In another embodiment, the PV is bovine papillomavirus-1 (BPV-1) and the residues 36-49 have a peptide sequence of SEQ ID NO:1. In yet another embodiment, the PV is human papillomavirus (HPV). The molecule can be a macromolecule or a small molecule. In a preferred embodiment, the macromolecule is an antibody, or an antigen-binding fragment thereof. The antibody can be polyclonal or monoclonal. Preferably, the antibody is humanized.

In yet another embodiment, the present invention discloses a composition for generating an antibody that binds specifically to residues 36-49 of a papillomaviruses (PV) minor capsid L2, the composition comprising a peptide comprising residues 36-49 of a papillomaviruses (PV) minor capsid L2. In a preferred embodiment, the peptide is conjugated to keyhole-limpet hemocyannine (KLH) at the N-terminal via a cysteine moeity.

In yet another further embodiment, the present invention discloses an isolated antibody, or antigen-binding fragment thereof, that binds specifically to residues 36-49 (SEQ ID NO:1) of bovine papillomavirus-1 (BPV-1) minor capsid L2. The antibody or the antigen-binding fragment is capable of inhibiting infection of a mammalian cell by bovine papillomavirus-1 (BPV-1) or human papillomavirus 16 (HPV-16).

### EXAMPLES

### Example 1

### L2 Peptides and Antibodies

The following peptides were made by ADI (Dallas, Tex.): 1) WTP15L2-CDTIADKILKFGGLA corresponding to residues 36-49 of BPV-1 L2 (SEQ ID NO:1) with a cysteine moiety at the N-terminal, and 2) SCR15L2CIDGLGKLATIDAKF (SEQ ID NO:2) corresponding to the same BPV-1 L2 residues, with the cysteine moiety at the N-terminal, in random order. The peptides were resuspended in serum-free DMEM as per manufacturer's instruction at 10 mM concentration. The proper amount of peptide from stock was added to the cultures to attain the final concentrations in the neutralization blocking experiments. Antibodies to the L2 residues 36-49 were made by ADI after conjugation of the 36-49 amino acids peptide to keyhole-limpet hemocyanine (KLH). Two rabbits were immunized and bleeds were collected at 4 week intervals. Pre-immune serum was collected from both rabbits.

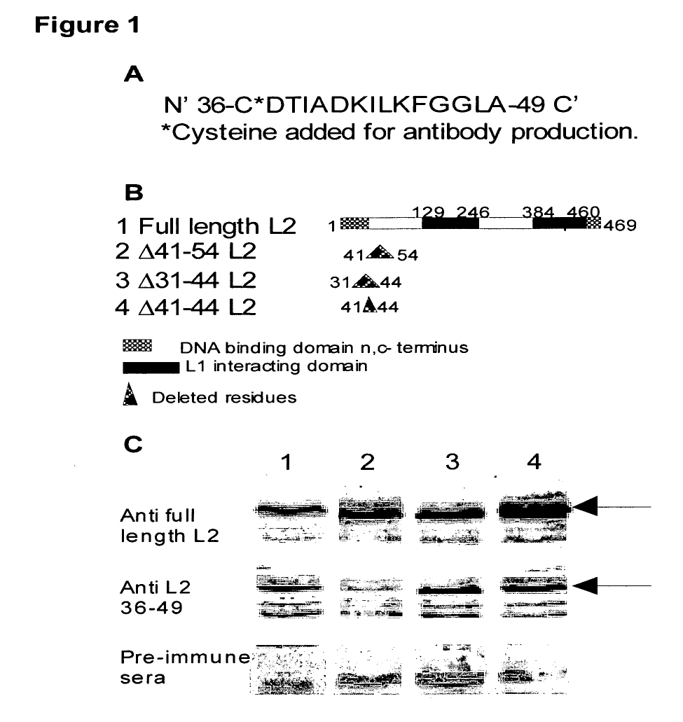

Antibodies were generated in the two rabbits using the peptide corresponding to BPV-1 L2 residues 36-49 conjugated to KLH by the addition of a cysteine at the n-terminus of the peptide (FIG. 1A). In order to determine the antibody's specificity of L2-binding, BPV-1 L2 lysates corresponding to full-length L2 and L2 deletion mutants were made from transfected Cos-7 cells. FIG. 1B depicts the 469 residues of L2, shown are the described regions of DNA binding at the n-terminus residues 1-30 and the two L1 interacting domain for capsid formation residues 129-246 and 384-460 (17, 44). Also shown in this figure are the residues deleted from the mutant constructs (FIG. 1B), these correspond to amino acids 41-54, 31-44, or 41-44 (FIG. 1B lanes 2-4 respectively). The constructs were made using standard PCR techniques as previously described (4). Lysates were run on a 10% resolving gel and blotted using the following three antibodies: 1) BPV-1 antibody BL2 made against full length BPV-1 L2 (kindly provided by Richard Roden) (FIG. 1C, top panel), 2) the antibody we generated against residues 36-49 (FIG. 1C, middle panel), and 3) the pre-immune (PI) sera from the injected rabbit (FIG. 1C, bottom panel). As shown in FIG. 1C, the various L2 constructs are expressed equally well as compared to the full length L2, and are all recognized by the antibody generated to full-length L2 (FIG. 1C, top panel). The antibody generated against residues 36-49 detects the 62 kDa full-length capsid protein (FIG. 1C, middle panel, lane 1) and detects both L2 mutants deleted of residues 31-44, and 41-44 (FIG. 1C, middle panel lanes 3, and 4 correspondingly). There is loss of antibody detection of L2 with the deletion of amino acids 41-54 (FIG. 1C, middle panel lane 2). There is no detection of L2 using pre-immune sera (FIG. 1C, lower panel).

### Example 2

### Pseudovirion Production and Purification

The bicistronic plasmid pShell carrying the BPV-1 L1 and L2 sequences, the GFP cDNA containing plasmid 8fwb, and the 293TT viral packaging cell line were a generous gift from Drs. Day and Schiller (6). Pseudovirus production was performed as described (6). In brief: 293TT cells at 70-80% confluence in a T175 cm² flask were co-transfected with 15 ug of pShell and 15 ug of 8fwb, in 65 ul of lipofectamine 2000 (Invitrogen, Carlsbad, Calif.) as per manufacturer's instructions. After 24 hours, the cells were trypsinized and split into two new T175 cm² flasks. After 24 hours, the cells were harvested by centrifugation and re-suspended in 500 ul of cell lysis buffer (Brij, benzonase, DNAsafe, in PBS) and incubated at 37° C. with gentle rotation for 16 hours. The virus preparation was put on ice for 5 minutes before the addition of 85 ul of 5M NaCl. After a 20 minute incubation on ice with inversion every 5-10 minutes, the preparations were spun at 4° C. in a microcentrifuge for 10 minutes at 5,000 rpms. The supernatant was then layered over a gradient of optiprep consisting of a lower layer of 39%, a middle layer of 37%, and an upper layer of 33% that had been left to equilibrate for one hour. The tubes were spun in a SW55Ti Beckman centrifuge for 3½ hours at 50,000 rpm (234,000×g) at 16° C. After the spin, 400 ul fractions were collected and tested for the presence of virus using FACS analysis of Cos-7 infected cells. A second purification step was performed on the virus preparations used for electron microscopic (EM) studies. For this purification, the layers of optiprep used were 50%, 39%, and 37%. Fractions of 200 ul were collected and tested for the presence of the virus used for EM.

### Example 3

### Determining Viral Titer

Viral titer was performed on Cos-7 cells. In brief, 50,000 Cos-7 cells/well were plated on a 24 well plate for 24 hours. At 24 hours, cells were washed in cold media (DME-10% FBS) and incubated on ice for 10 minutes in cold media. After 10 minutes cold media was added with 2, 5, or 8 ul of virus to the wells and the plate was incubated on ice for 2 hours. After two hours, the cells were washed ×3 in DME-10% FBS at room temperature, and incubated at 37° C. in 500 ul DME-10% FBS for 48 hours. Cells were then harvested and analyzed for GFP fluorescence by FACS analysis. 10,000 cells were counted and the percentage of green cells was used to determine the titer per ml of virus. Real time-PCR was performed on the DNA extracted from the virus preparation as described previously using GFP primers (6). The number of viral particles needed to obtain one infectious viral particle, i.e., a green cell, was determined by dividing the total number of viral particles obtained from the real-time PCR by the number of GFP positive cells obtained by FACS. We had a consistent 140-150 viral particles/green cell.

### Example 4

### Affinity Purification of L2 Peptide Antibody

Affinity purification of antibody derived from the rabbits immunized with the BPV-1 L2 peptide corresponding to residues 36-49 was performed using a Hi-trap NHS column (GE Healthcare, Piscataway, N.J.). Following manufacturers' instructions, column was washed in 1 mM HCl and washed with 10 volumes of coupling buffer (200 mM NaHCO3, 500 mM NaCl). 500 ul of 10 mM peptide was then passed through the column using a 10 cc syringe and circulated for an hour. Cross-linking of the peptide to the column was followed by a brief incubation period of the column at room temperature and then extensive washing with 18 volumes of buffer A (500 mM ethanolamine, 500 mM NaCl pH 8.3) and buffer B (100 mM NaOAc, 500 mM NaCl pH 4.0) alternating 6 volumes of each at a time. Once ligand was bound, 10 mls of sera diluted 1:3 in PBS were circulated through the column for a period of 6 hours on ice. Column was then washed with 20 volumes of ice cold PBS. Bound antibody was eluted from the column using elution buffer (50 mM glycine, 150 mM NaCl) at pH 2.5. Eight 500 ul fractions were collected and their pH was adjusted to around pH 7 using 1/10 volume of 1.5 mM Tris pH 8.8. BCA protein assays were performed to determine the peak and concentration of purified IgG. Western blots were used to determine that the L2 specific antibody was indeed purified.

### Example 5

### Western Blots

Cos-7 cells were transfected using lipofectamine 2000 as per manufacturer's instructions. After 24 hour incubation, cells were harvested in ice cold RIPA buffer (1% sodium deoxycholate, 0.1% SDS, 1% Triton X-100, 1% bovine hemoglobin, 1 mM iodoacetamide, 10 mM Tris HCl pH 8.0, 140 mM NaCl, 0.025% NaN3) containing protease inhibitors (GE Healthcare). The lysates were spun at 13,200 rpms for 5 minutes at 4° C. in a microcentrifuge. The supernatant was transferred to a new tube and frozen at −20° C. until used. Samples were run on a 10% SDS-PAGE gel, transferred to nitrocellulose, and blocked in 5% dry milk. Blocking, washing of the blots (3× between any step), and antibody incubations were all done in TNET (50 mM Tris HCl pH 7.4, 150 mM HCl, 5 mM EDTA, 0.5% triton-X 100). Primary antibodies for western analysis were used at 1:1000. Incubation of antibody was for a minimum of 3 hours at room temperature, to a maximum of overnight at 4° C. Fluorescent secondary antibodies used were used at 1:20,000 dilution for 30 minutes at room temperature. Blots were scanned in an Odyssey imaging system (Odyssey, Lincoln, Nebr.). Data was analyzed using Odyssey provided software.

### Example 6

### Neutralization Experiments

Virus was incubated with appropriate amount of affinity purified antibody for 1 hour on ice. The antibody-virus mix was then added onto cells that were chilled on ice for a minimum of 10 minutes. Infected cells were then incubated on ice for 2 hours incubation at 37° C. for the described times. Infection levels were measured by FACS after cells were harvested. 10,000 cells were counted by FACS; the number of GFP positive cells was used to determine percent infection.

### Example 7

### Immunofluorescence

Infected cells grown on glass coverslips (Fisher Scientific, cat #1254580) were fixed in 3% PFA for 10 minutes at 4° C. at the appropriate time points. Coverslips were treated as follows after fixation: cells were permeabilized in immunofluorescence buffer (0.2% fish skin gelatin, 0.2% TritonX-100 in phosphate-buffered saline [PBS]) for 10 min, followed by 1-hr incubation in immunofluorescence buffer with the appropriate primary antibodies. Antibody working dilutions were: mouse-anti L1 5B6 at 1:100 (generously provided by Dr. Roden, Johns Hopkins, MD), goat-anti LAMP1 at 1:100 (Santa Cruz, Santa Cruz, Calif.), mouse-anti L1 1H8 at 1:50 (GeneTex, San Antonio, Tex.), and goat-anti eeA1 at 1:100 (Santa Cruz). Cells were then washed three times in PBS. Fluorescently labeled Alexa-fluor donkey-anti-rabbit 488, donkey anti-rabbit 594, chicken anti-goat 488 and donkey-anti-mouse 594, (Molecular Probes, Eugene, Oreg.) were used as secondary antibodies in 30-min incubation in immunofluorescence buffer at 1:2,000 dilution. TOPRO-3 (Invitrogen) was added to the secondary antibody mixture at a 1:1000 dilution for nuclear staining. After washing in PBS ×3, coverslips were mounted on glass slides using Prolong anti-fade mounting medium (Invitrogen). All antibody incubations were performed at room temperature.

Fluorescence confocal microscopy was performed with an Olympus Fluoview 300 microscope with Fluoview software (Olympus, Melville, N.Y.).

### Example 8

### Negative Staining of Viral Particles for Electron Microscopy

Double purified virus-like particles were pre-incubated with affinity purified antibody against L2 residues 36-49 and with affinity purified glutathione-s-transferase (ADI) as an isotypic control for one hour on ice. Carbon filmed nickel grids (Electron Microscopy Sciences, Hatfield, Pa.) were then placed on 30 ul droplets of the mixture for 15 minutes, washed in blocking buffer (PBS, 2% albumin), and placed on 30 ul droplets of secondary anti-rabbit 10 nM immunogold labeled antibody (Sigma-Aldrich, St. Louis, Mo.). Grids were then fixed with 2% paraformaldehyde for 5 minutes, washed, and negatively stained using 2% phosphotungstitic acid. Analysis was performed at Rosalind Franklin University of Science and Medicine on a JEOL JEM-1230 transmission electron microscope at an accelerating velocity of 80 kV.

### Example 9

### Other Reagents

Syntaxin 18 construct was provided by Dr. Tagaya (Tokyo University, Tokyo Japan) (29). M2 Flag beads were purchased from Sigma. L2 mutant constructs were previously described (4). L2 model was obtained from http://robetta.bakerlab.org/. Control DNA vector control pA3M was a gift from Dr. Robertson (Univ. of Penn., Philadelphia, Pa.).

### Example 10

### Residues 36-49 are Exposed on the Outer Surface of BPV-1 Pseudovirions

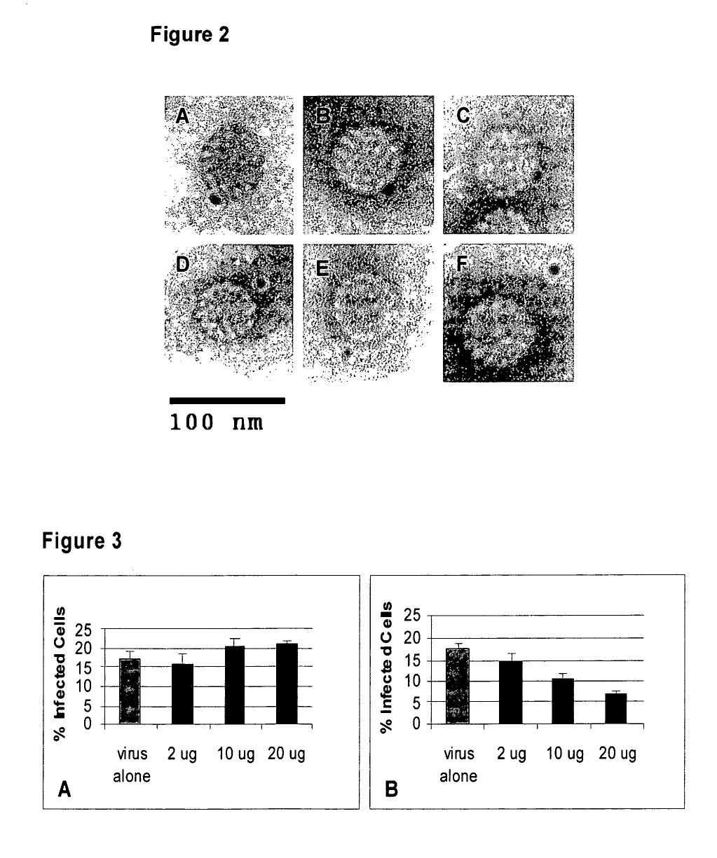

In order to determine the location of the L2 residues 36-49 on the viral capsid, we performed immuno-electron microscopy with affinity purified antibody generated against residues 36-49 (FIG. 2 A-C). Using anti-rabbit gold-labeled secondary antibodies, we were able to detect the binding of the antibody against residues 3649 on the surface of viral particles (FIG. 2 A-C). The viral particles shown have the expected size of 50 nm in diameter, while the secondary antibody conjugated to gold particles is 10 nm in diameter and is visualized here as the smaller black dots due to their higher density after negative staining with 2% phosphotungstitic acid. We performed the same analysis with an isotypic control antibody generated against the GST protein, but were unable to find any gold labeled particles bound to virions (FIG. 2 D-F).

### Example 11

### Affinity Purified Antibody to BPV-1 L2 Residues 36-49 Neutralizes Infection in a Dose Dependent Manner

Our finding that the antibody to residues 36-49 was able to bind to the surface of the virions prompted us to explore the potential of this antibody to neutralize infection. Since our antibody was rabbit affinity purified, another affinity purified rabbit antibody was obtained from the same company that generated the antisera in order to serve as an isotypic control. BPV-1 pseudovirions containing the GFP cDNA containing plasmid 8fwb were pre-incubated with antibody for 1 hour on ice and then added to 100,000 Cos-7 cells. Our viral titer was based on GFP positive cells and experiments were performed with sufficient virus to infect 20,000 cells or a titre of 2×10⁴ infectious units. Infections were harvested for analysis by flow cytometry. The percentage of cells infected (i.e., GFP positive) with pseudovirus alone was 17.4% (FIG. 3A, B gray bar). The percentage of cells infected with virus that was pre-incubated with increasing concentrations of the isotypic affinity purified anti-GST control antibody was not decreased (FIG. 3A black bars). The addition of 2 ug, 10 ug, or 20 ug of anti-GST antibody had levels of infection corresponding to 16.1%, 20.6% and 21.6%, respectively, which is not a statistically significant change as compared to virus alone. In contrast, the addition of increasing amounts of affinity purified antibody generated against residues 36-49 demonstrates a dose-dependent decrease in the percentage of cells infected with the pseudovirion (FIG. 3B black bars). Compared to virus alone, in the presence of 2 ug, 10 ug, and 20 ug of the antibody against the 15 amino acid residue of BPV-1 L2 there is a 14.5%, 10.9%, and 7.3% level of infection, which is nearly a 60% decrease in infection. Each bar represents the average of three experiments in which 10,000 events were counted and error bars demonstrate the standard deviation in the experiments.

### Example 12

### Neutralization of Infection with Affinity Purified Antibody is Inhibited by a Peptide of Residues 36-49 in a Dose Dependant Manner

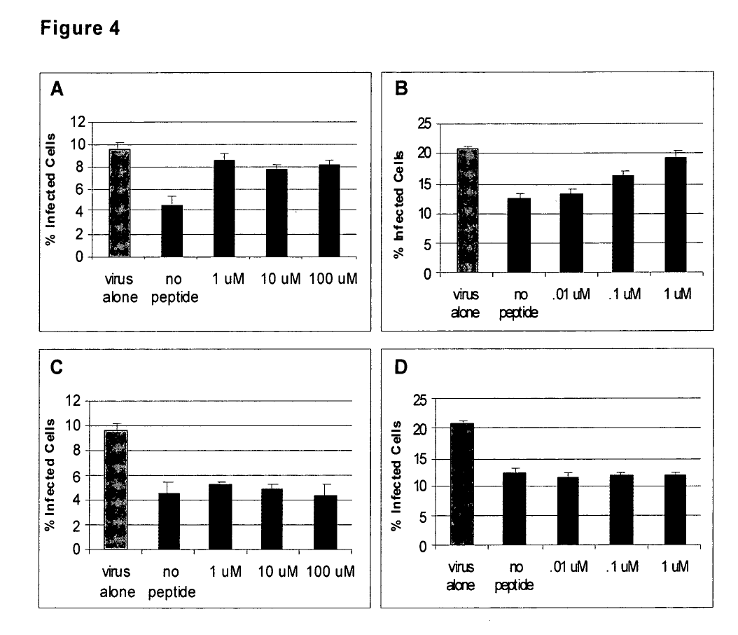

In order to demonstrate the specificity and reversibility of inhibition of infection by the antibody to the conserved 15 residue region of the capsid protein L2, we performed a competition experiment with the peptide corresponding to L2 residues 36-49 and a control peptide of the same residues in scrambled order (FIG. 4). The neutralization data showed that 20 ug of affinity purified antibody decrease the level of infection by greater than 60% (FIG. 3B). Increasing concentrations of peptide were mixed with 20 ug of affinity purified antibody prior to the 2 hour infection. Our first experiment was done with peptide concentrations ranging from 1 uM to 100 uM (FIG. 4 A, C). As before, neutralization of infection was observed with the addition of the purified antibody corresponding to a 50-60% decrease in infection as compared to virus alone (FIG. 3, A, C compare no peptide bars, with gray virus alone bars). The addition of 1 uM of the peptide corresponding to residues 36-49 in the correct sequence resulted in complete loss of this neutralization (FIG. 3A 1 uM bar). The addition of 10 uM or 100 uM of this peptide also blocked inhibition of infection (FIG. 3A 10 and 100 uM bars). In contrast, the addition of 1 uM, 10 uM, or 100 uM of the scrambled peptide had no affect on infection (FIG. 1 C, 1, 10, 100 uM bars). Since the inhibition of the antibody neutralization was virtually complete at 1 uM peptide, a dose-dependent effect was not observed. Thus, we analyzed the inhibition of neutralization by incubating the antibody with 10 nM, 100 mM, and 1 uM of the peptides (FIG. 4 B, D). Once again, the inhibition of the antibody's neutralizing effect was nearly complete at 1 uM wild-type peptide (FIG. 4B, 1 uM bar), but this time we observed a dose-dependent inhibition of infection with 10 nM, 100 nM, and 1 uM of the wild-type peptide (FIG. 4B 0.01, 0.1, and 1 uM bars). There was a 6.9% loss of neutralization at 10 nM, and 45% loss at 100 uM. We again did not observe any loss of infection when the affinity purified antibody was incubated with the scrambled peptide at concentrations ranging from 10 nM to 1 uM (FIG. 4D, 0.01, 0.1, and 1 uM bars).

### Example 13

### Incubation of Virus with the Neutralizing Affinity Purified Antibody does not Interfere with the Virions Ability to Bind to Cells, nor Blocks the Initial Endocytosis of the Virus

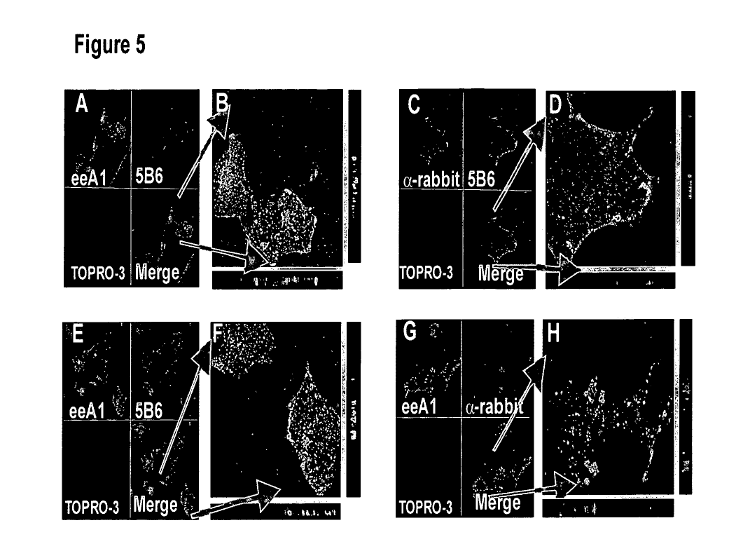

To determine if the observed neutralization was due to the antibody blocking the binding of the virus to the surface of the cells or the antibody preventing the internalization of the virus, immunofluorescence analysis of infection was performed at the cellular level. Our electron microscopy data showed that the antibody against residues 36-49 was able to bind to the outside of the virus. This binding allowed us to detect viral particles/antibody complexes in cells by staining cells with a fluorescent labeled anti-rabbit antibody (FIG. 5, α-rabbit images). We used anti-L1 antibody 5B6 to stain the intact viral particle at 5 minutes and eeA1 to identify early endosomes (FIG. 5, 5B6 and eeA1 images, respectively). Nuclear DNA was stained with TOPRO-3 (FIG. 5, TOPRO-3 panels). At 5 minutes post-infection, co-localization is noted between the virus stained with 5B6 and the early endosomes stained with eeA1, consistent with receptor mediated clathrin dependent endocytosis (FIG. 5 A, B). FIG. 5B represents an enlarged portion of a z-stack series that was taken of the image in 5A, allowing a multiplane dissection of a cell so that signal overlaps can be definitively determined. The yellow overlap of the staining of 5B6 and eeA1 is observed on all three planes. This data confirms that the pseudovirions enter the cell in a clathrin-dependent manner as has been described (16). In order to show that the antibody generated against residues 36-49 recognized viral particles, we performed immunofluorescence analysis of cells infected with virus that was pre-incubated, and thus, bound by the antibody against residues 36-49 prior to infection. The staining of 5B6 overlaps in fluorescence with the signal obtained with the anti-rabbit antibody that recognizes the virus bound antibody against residues 36-49 (FIGS. 5, C, and D). In FIG. 5 D (the z-stack) we show the yellow overlap of both signals labeling of the pseudovirions. The co-localization of 5B6 and eeA1 (FIG. 5B z-stack), is still observed in experiments where incubation of virus with the affinity purified antibody against 36-49 precedes infection (FIG. 5 E, F). In these cells, pre-incubation results in loss of infection. We also observed the co-localization of staining of eeA1 with the staining of the bound anti-L2 antibody (FIG. 5 G, F). The staining of virus pre-incubated with the affinity purified antibody against residues 36-49 with eeA1 suggested that although neutralization is achieved with the affinity purified antibody against residues 36-49, the virus is still able to be internalized into endosomes.

### Example 14

### Incubation of Virus with the Neutralizing Affinity Purified Antibody does not Interfere with the Virus Ability to Reach LAMP-1 Positive Vesicles after 2 or 4 Hours Post-Infection

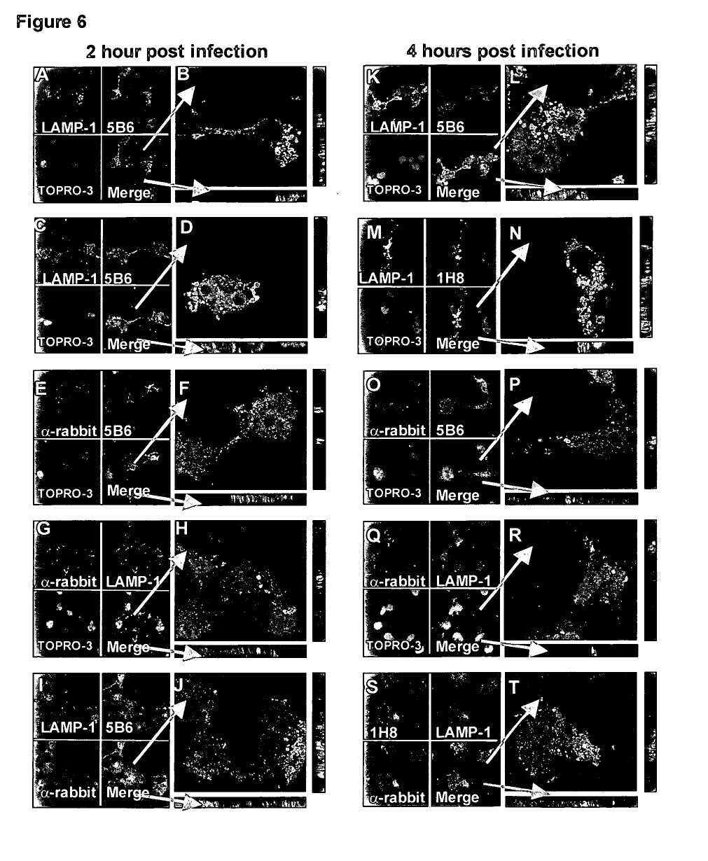

We further examined the trafficking of the virus incubated with the neutralizing antibody against residues 36-49 by co-staining with the late endosome and lysosome marker LAMP-1. A second anti-L1 antibody was tested at 4 hours since the epitope recognized by 5B6 is conformation dependent, and has been shown to be largely lost at 4 hours (16). The anti-L1 1H8 antibody neutralizes infection and recognizes a linear L1 epitope on BPV-L1 virions regardless of capsid integrity. There is overlap of LAMP-1 and 5B6 staining in cells infected with pseudovirions at 2 and 4 hours, although there is less 5B6 staining observed at 4 hours suggesting loss of the epitope (FIGS. 6 A and B 2 hours, K and L 4 hours). Cells that were infected with pseudovirions that were incubated with the neutralizing antibody against residues 36-49 had overlapping staining of 5B6 and 1H8 at 2 hours, and overlapping signal of LAMP-1 and 1H8 at 4 hours (FIGS. 6 C and D 2 hours, M and N 4 hours). There was more staining observed with the anti L1 antibody 1H8 than with the anti-L1 antibody 5B6 at 4 hours (FIG. 6, compare L and N). We were again able to see the overlap of the pseudovirus bound antibody to residues 36-49 and 5B6 at 2 and 4 hours (FIGS. 6 E and F 2 hours, O and P 4 hour). There appears to be more staining with 5B6 observed at 4 hours when pseudovirions are bound with the antibody against residues 36-49 (FIG. 6, compare P and L). Staining for LAMP-1 and the bound antibody to residues 36-49 also showed overlap in signals at 2 and 4 hours (FIGS. 6, G and H 2 hours, Q and R 4 hours). Staining of cells for LAMP-1, 5B6 and the bound antibody shows that these three signals overlap at 2 hours (FIGS. 6 I and J, white dots on merge). Finally, staining of infected cells for LAMP-1, the bound antibody to residues 36-49, and anti-L1 1H8 antibody shows that the three signals give the white overlapping signal of all three antibodies (FIGS. 6, S and T white dots on merge. These data demonstrate that the pseudovirions traffic through a LAMP-1 positive vesicle at 2 and 4 hours, and that the neutralizing antibody does not prevent this trafficking from occurring.

### Example 15

### Antibody to BPV-1 L2 Residues 36-49 Interferes with the Co-Immunoprecipitation of BPV-1 L2 and Syntaxin 18

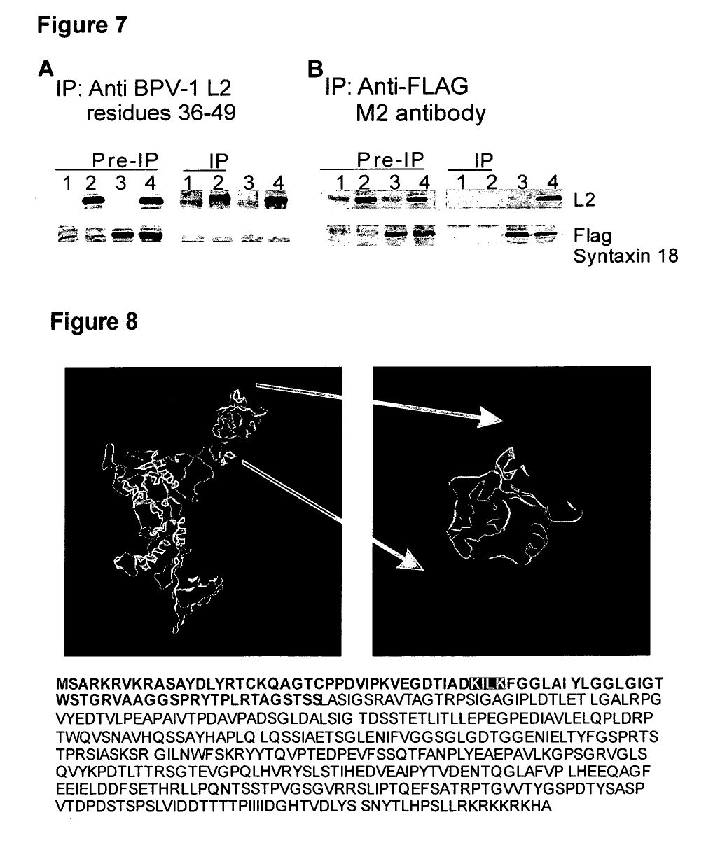

Our laboratory had previously shown that BPV-1 L2 and Flag-syntaxin 18 co-immunoprecipitated when co-transfected (4). Our control experiments again demonstrate the following: 1) Flag-syntaxin 18 can co-immunoprecipitate with untagged BPV-1 L2 (FIG. 7 B, lanes 4), 2) the M2-FLAG antibody bound to sepharose beads does not precipitate L2 non-specifically (FIG. 7B lane 2), and 3) did not precipitate a non-specific band at the size of BPV-1 L2 in vector control transfected cells (FIG. 7B lane 1). The M2-FLAG coated beads did immunoprecipitating BPV-1 L2 and Flag-syntaxin 18 with the antibody raised against residues 36-49 were unsuccessful (FIG. 7A, lane 4). The antibody against L2 residues 36-49 did not immunoprecipitate or co-immunoprecipitate Flag-syntaxin 18 (FIG. 7A lanes 3 and 4), nor was there a non-specific band at the size of BPV-1 L2 immunoprecipitated from vector control transfected cells (FIG. 7A lanes 1 and 3). BPV-1 L2 was immunoprecipitated specifically with the antibody against residues 36-49 (FIG. 7A lanes 2 and 4). Antibody to full length BPV-1 L2 (gift from Richard Roden) and anti Flag-M2 antibody (Sigma) were used to detect the proteins in western blots.

### Example 16

### HPV 16 Pseudovirion Infection is Neutralized with the Affinity Purified Antibody Made Against Residues 36-49

293TT were infected with 1 or 2 ul of HPV 16 pseudovirions, approximately 7×10⁴ infectious units/ul. Addition of 100 ug of affinity purified antibody against BPV-1 L2 inhibits infection by 35% (FIG. 10).

HPV 16 residues 36-49 KTIADQILQYGSMG are modified by the addition of N-terminal cysteine in order to couple the peptide to keyhole-limpet hemocyanin, a common antigen used when developing novel antibodies due to its strong antigenic, immunogenic induction capabilities (4ADI Inc., Dallas, Tex.). Antibody has been IgG purified and affinity purified.

TABLE 1

| | BPV1 | HPV5 | HPV18 | HPV31 | HPV45 |
|---|---|---|---|---|---|
| Virus alone | 9.84 | 2.62 | 60.92 | 4.11 | 12.86 |
| Virus + control IgG | 4.23 | 2.29 | 41.59 | 2.48 | 8.71 |
| Virus + 16L2 | 2.58 | 2.16 | 28.78 | 1.55 | 5.59 |
| 36-49 IgG | | | | | |
| % block | 73.78 | 17.56 | 52.76 | 62.29 | 56.53 |

Table 1 shows other genotype infection levels of COS-7 cells in the presence of the HPV16 L2 antibody 36-49. COS-7 cells were infected with 20 ug of immune IgG. The data shows block of infection across several genotypes. The present invention contemplates normalizing this experiment with further control. Observations of neutralization with control IgG are noted, although to a lesser extent than with the immune Frac30 IgG.

While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. The appended claims should be construed broadly and in a manner consistent with the spirit and the scope of the invention herein. It is understood that, given the above description of the embodiments of the invention, various modifications may be made by one skilled in the art. Such modifications are intended to be encompassed by the claims below.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### REFERENCES

1. Baker, T. S., W. W. Newcomb, N. H. Olson, L. M. Cowsert, C. Olson, and J. C. Brown. 1991. Structures of bovine and human papillomaviruses. Analysis by cryoelectron microscopy and three-dimensional image reconstruction. Biophys J 60:1445-56.
2. Bordeaux, J., S. Forte, E. Harding, M. S. Darshan, K. Klucevsek, and J. Moroianu. 2006. The 12 minor capsid protein of low-risk human papillomavirus type 11 interacts with host nuclear import receptors and viral DNA. J Virol 80:8259-62.
3. Bosch, F. X., M. M. Manos, N. Munoz, M. Sherman, A. M. Jansen, J. Peto, M. H. Schiffman, V. Moreno, R. Kurman, and K. V. Shah. 1995. Prevalence of human papillomavirus in cervical cancer: a worldwide perspective. International biological study on cervical cancer (IBSCC) Study Group. J Natl Cancer Inst 87:796-802.
4. Bossis, I., R. B. Roden, R. Gambhira, R. Yang, M. Tagaya, P. M. Howley, and P. I. Meneses. 2005. Interaction of tSNARE Syntaxin 18 with the Papillomavirus Minor Capsid Protein Mediates Infection. J Virol 79:6723-31.
5. Bousarghin, L., A. Touze, P. Y. Sizaret, and P. Coursaget. 2003. Human papillomavirus types 16, 31, and 58 use different endocytosis pathways to enter cells. J Virol 77:3846-50.
6. Buck, C. B., D. V. Pastrana, D. R. Lowy, and J. T. Schiller. 2004. Efficient intracellular assembly of papillomaviral vectors. J Virol 78:751-7.
7. Buck, C. B., D. V. Pastrana, D. R. Lowy, and J. T. Schiller. 2005. Generation of HPV pseudovirions using transfection and their use in neutralization assays. Methods Mol Med 119:445-62.
8. Burd, E. M. 2003. Human papillomavirus and cervical cancer. Clin Microbiol Rev 16:1-17.
9. Campo, M. S. 2002. Animal models of papillomavirus pathogenesis. Virus Res 89:249-61.
10. Christensen, N. D., and J. W. Kreider. 1991. Neutralization of CRPV infectivity by monoclonal antibodies that identify conformational epitopes on intact virions. Virus Res 21:169-79.
11. Crum, C. P., M. Symbula, and B. E. Ward. 1989. Topography of early HPV 16 transcription in high-grade genital precancers. Am J Pathol 134:1183-8.
12. Culp, T. D., N. M. Cladel, K. Balogh, L. R. Budgeon, A. Mejia, and N. D. Christensen. 2006. Papillomavirus particles assembled in 293TT cells are infectious in vivo. J. Virol.
13. Darshan, M. S., J. Lucchi, E. Harding, and J. Moroianu. 2004. The 12 minor capsid protein of human papillomavirus type 16 interacts with a network of nuclear import receptors. J Virol 78:12179-88.
14. Database, H. 1994-1997. The Human Papillomaviruses Compendium On Line. hpv-web.lanl.gov/stdgen/virus/hpv/compendium/htdocs/HTML_FILES/HPVcompintro4.html#comp97.
15. Day, P. M., C. C. Baker, D. R. Lowy, and J. T. Schiller. 2004. Establishment of papillomavirus infection is enhanced by promyelocytic leukemia protein (PML) expression. Proc Natl Acad Sci USA.
16. Day, P. M., D. R. Lowy, and J. T. Schiller. 2003. Papillomaviruses infect cells via a clathrin-dependent pathway. Virology 307: 1-11.
17. Day, P. M., R. B. Roden, D. R. Lowy, and J. T. Schiller. 1998. The papillomavirus minor capsid protein, L2, induces localization of the major capsid protein, L1, and the viral transcription/replication protein, E2, to PML oncogenic domains. J Virol 72:142-50.
18. de Sanjose, S., and J. Palefsky. 2002. Cervical and anal HPV infections in HIV positive women and men. Virus Res 89:201-11.
19. Deau, M. C., M. Favre, and G. Orth. 1991. Genetic heterogeneity among human papillomaviruses (HPV) associated with epidermodysplasia verruciformis: evidence for multiple allelic forms of HPV5 and HPV8 E6 genes. Virology 184:492-503.
20. Doorbar, J., and P. H. Gallimore. 1987. Identification of proteins encoded by the L1 and L2 open reading frames of human papillomavirus 1a. J Virol 61:2793-9.
21. Evander, M., I. H. Frazer, E. Payne, Y. M. Qi, K. Hengst, and N. A. McMillan. 1997. Identification of the alpha6 integrin as a candidate receptor for papillomaviruses. J Virol 71:2449-56.
22. Fay, A., W. H. t. Yutzy, R. B. Roden, and J. Moroianu. 2004. The positively charged termini of L2 minor capsid protein required for bovine papillomavirus infection function separately in nuclear import and DNA binding. J Virol 78:13447-54.
23. Florin, L., K. A. Becker, C. Lambert, T. Nowak, C. Sapp, D. Strand, R. E. Streeck, and M. Sapp. 2006. Identification of a dynein interacting domain in the papillomavirus minor capsid protein 12. J Virol 80:6691-6.
24. Garcia-Pineres, A. J., A. Hildesheim, M. Trivett, M. Williams, L. Wu, V. N. Kewalramani, and L. A. Pinto. 2006. Role of DC-SIGN in the activation of dendritic cells by HPV-16 L1 virus-like particle vaccine. Eur J Immunol 36:437-45.
25. Gaukroger, J. M., L. M. Chandrachud, B. W. O'Neil, G. J. Grindlay, G. Knowles, and M. S. Campo. 1996. Vaccination of cattle with bovine papillomavirus type 4 L2 elicits the production of virus-neutralizing antibodies. J Gen Virol 77 (Pt 7):1577-83.
26. Ghys, P. D., C. Jenkins, and E. Pisani. 2001. HIV surveillance among female sex workers. Aids 15 Suppl 3:S33-40.
27. Gissmann, L., H. Pfister, and H. Zur Hausen. 1977. Human papilloma viruses (HPV): characterization of four different isolates. Virology 76:569-80.
28. Hagensee, M. E., J. E. Cameron, J. E. Leigh, and R. A. Clark. 2004. Human papillomavirus infection and disease in HIV-infected individuals. Am J Med Sci 328:57-63.
29. Hatsuzawa, K., H. Hirose, K. Tani, A. Yamamoto, R. H. Scheller, and M. Tagaya. 2000. Syntaxin 18, a SNAP receptor that functions in the endoplasmic reticulum, intermediate compartment, and cis-Golgi vesicle trafficking. J Biol Chem 275:13713-20.
30. Hatsuzawa, K., T. Tamura, H. Hashimoto, H. Hashimoto, S. Yokoya, M. Miura, H. Nagaya, and I. Wada. 2006. Involvement of syntaxin 18, an endoplasmic reticulum (ER)-localized SNARE protein, in ER-mediated phagocytosis. Mol Biol Cell 17:3964-77.
31. Holmgren, S. C., N. A. Patterson, M. A. Ozbun, and P. F. Lambert. 2005. The minor capsid protein L2 contributes to two steps in the human papillomavirus type 31 life cycle. J Virol 79:3938-48.
32. IARC. 2005. http://www.iarc.fr/. 2005.
33. Joyce, J. G., J. S. Tung, C. T. Przysiecki, J. C. Cook, E. D. Lehman, J. A. Sands, K. U. Jansen, and P. M. Keller. 1999. The L1 major capsid protein of human papillomavirus type 11 recombinant virus-like particles interacts with heparin and cell-surface glycosaminoglycans on human keratinocytes. J Biol Chem 274:5810-22.
34. Kamper, N., P. M. Day, T. Nowak, H. C. Selinka, L. Florin, J. Bolscher, L. Hilbig, J., T. Schiller, and M. Sapp. 2006. A membrane-destabilizing peptide in capsid protein L2 is required for egress of papillomavirus genomes from endosomes. J Virol 80:759-68.
35. Kawana, K., H. Yoshikawa, Y. Taketani, K. Yoshiike, and T. Kanda. 1999. Common neutralization epitope in minor capsid protein L2 of human papillomavirus types 16 and 6. J Virol 73:6188-90.
36. Kawana, K., H. Yoshikawa, Y. Taketani, K. Yoshiike, and T. Kanda. 1998. In vitro construction of pseudovirions of human papillomavirus type 16: incorporation of plasmid DNA into reassembled L1/L2 capsids. J Virol 72:10298-300.
37. Klucevsek, K., J. Daley, M. S. Darshan, J. Bordeaux, and J. Moroianu. 2006. Nuclear import strategies of high-risk HPV18 L2 minor capsid protein. Virology 352:200-8.
38. Knipe D M, H. P. (ed.). 2001. Fundamental virology, 4th ed. Lippincott Williams & Wilkins, Philadelphia, Pa.
39. Kondo, K., Y. Ishii, H. Ochi, T. Matsumoto, H. Yoshikawa, and T. Kanda. 2006. Neutralization of HPV16, 18, 31, and 58 pseudovirions with antisera induced by immunizing rabbits with synthetic peptides representing segments of the HPV16 minor capsid protein L2 surface region. Virology.
40. Koutsky, L., Wright T., Ebel C. 2004. HPV in 2004 Public Health Response to New Tools. CDC National STD Prevention Conference.
41. Liu, W. J., L. Gissmann, X. Y. Sun, A. Kanjanahaluethai, M. Muller, J. Doorbar, and J. Zhou. 1997. Sequence close to the N-terminus of L2 protein is displayed on the surface of bovine papillomavirus type 1 virions. Virology 227:474-83.
42. McMillan, N. A., E. Payne, I. H. Frazer, and M. Evander. 1999. Expression of the alpha6 integrin confers papillomavirus binding upon receptor-negative B-cells. Virology 261:271-9.
43. Muller, M., L. Gissmann, R. J. Cristiano, X. Y. Sun, I. H. Frazer, A. B. Jenson, A. Alonso, H. Zentgraf, and J. Zhou. 1995. Papillomavirus capsid binding and uptake by cells from different tissues and species. J Virol 69:948-54.
44. Okun, M. M., P. M. Day, H. L. Greenstone, F. P. Booy, D. R. Lowy, J. T. Schiller, and R. B. Roden. 2001. L1 interaction domains of papillomavirus 12 necessary for viral genome encapsidation. J Virol 75:4332-42.
45. Pagliusi, S. 2004. Researc on Bacterial Vaccines. World Health Organization. http://www.who.int/vaccine_research/diseases/hpv/en/.
46. Pastrana, D. V., R. Gambhira, C. B. Buck, Y. Y. Pang, C. D. Thompson, T. D. Culp, N. D. Christensen, D. R. Lowy, J. T. Schiller, and R. B. Roden. 2005. Cross-neutralization of cutaneous and mucosal Papillomavirus types with anti-sera to the amino terminus of L2. Virology.
47. Pelkmans, L., and A. Helenius. 2003. Insider information: what viruses tell us about endocytosis. Curr Opin Cell Biol 15:414-22.
48. Pisani, E., G. P. Garnett, N. C. Grassly, T. Brown, J. Stover, C. Hankins, N. Walker, and P. D. Ghys. 2003. Back to basics in HIV prevention: focus on exposure. Bmj 326:1384-7.
49. Pisani, E., S. Lazzari, N. Walker, and B. Schwartlander. 2003. HIV surveillance: a global perspective. J Acquir Immune Defic Syndr 32 Suppl 1:S3-11.
50. Pisani, P., D. M. Parkin, N. Munoz, and J. Ferlay. 1997. Cancer and infection: estimates of the attributable fraction in 1990. Cancer Epidemiol Biomarkers Prev 6:387-400.
51. Pyeon, D., P. F. Lambert, and P. Ahlquist. 2005. Production of infectious human papillomavirus independently of viral replication and epithelial cell differentiation. Proc Natl Acad Sci USA 102:9311-6.
52. Richards, R. M., D. R. Lowy, J. T. Schiller, and P. M. Day. 2006. Cleavage of the papillomavirus minor capsid protein, L2, at a furin consensus site is necessary for infection. Proc Natl Acad Sci USA 103:1522-7.
53. Roden, R. B., R. Kirnbauer, A. B. Jenson, D. R. Lowy, and J. T. Schiller. 1994. Interaction of papillomaviruses with the cell surface. J Virol 68:7260-6.
54. Roden, R. B., E. M. Weissinger, D. W. Henderson, F. Booy, R. Kimbauer, J. F. Mushinski, D. R. Lowy, and J. T. Schiller. 1994. Neutralization of bovine papillomavirus by antibodies to L1 and L2 capsid proteins. J Virol 68:7570-4.
55. Roden, R. B., W. H. t. Yutzy, R. Fallon, S. Inglis, D. R. Lowy, and J. T. Schiller. 2000. Minor capsid protein of human genital papillomaviruses contains subdominant, cross-neutralizing epitopes. Virology 270:254-7.
56. Selinka, H. C., T. Giroglou, T. Nowak, N. D. Christensen, and M. Sapp. 2003. Further evidence that papillomavirus capsids exist in two distinct conformations. J Virol 77:12961-7.
57. Selinka, H. C., T. Giroglou, and M. Sapp. 2002. Analysis of the infectious entry pathway of human papillomavirus type 33 pseudovirions. Virology 299:279-287.
58. Sibbet, G., C. Romero-Graillet, G. Meneguzzi, and M. S. Campo. 2000. alpha6 integrin is not the obligatory cell receptor for bovine papillomavirus type 4. J Gen Virol 81:327-34.
59. Sieczkarski, S. B., and G. R. Whittaker. 2002. Dissecting virus entry via endocytosis. J Gen Virol 83:1535-45.
60. Trus, B. L., R. B. Roden, H. L. Greenstone, M. Vrhel, J. T. Schiller, and F. P. Booy. 1997. Novel structural features of bovine papillomavirus capsid revealed by a three-dimensional reconstruction to 9 A resolution. Nat Struct Biol 4:413-20.
61. Unckell, F., R. E. Streeck, and M. Sapp. 1997. Generation and neutralization of pseudovirions of human papillomavirus type 33. J Virol 71:2934-9.
62. Volpers, C., F. Unckell, P. Schirmacher, R. E. Streeck, and M. Sapp. 1995. Binding and internalization of human papillomavirus type 33 virus-like particles by eukaryotic cells. J Virol 69:3258-64.
63. Zhou, J., D. J. Stenzel, X. Y. Sun, and I. H. Frazer. 1993. Synthesis and assembly of infectious bovine papillomavirus particles in vitro. J Gen Virol 74 (Pt 4):763-8.
64. zur Hausen, H., and E. M. de Villiers. 1994. Human papillomaviruses. Annu Rev Microbiol 48:427-47.

We claim:

1. An isolated antibody, or antigen-binding fragment thereof, that binds specifically to residues 36-49 of a papillomavirus (PV) minor capsid L2.

2. The antibody or the antigen-binding fragment of claim 1 used to inhibit infection of a mammalian cell by the papillomaviruses (PVs).

3. The antibody or the antigen-binding fragment of claim 1 wherein the PV is bovine papillomavirus.

4. The antibody or the antigen-binding fragment of claim 1 wherein the PV is bovine papillomavirus 1 (BPV-1) and wherein the residues 36-49 have a peptide sequence of SEQ ID NO:1.

5. The antibody or the antigen-binding fragment of claim 1 wherein the PV is human papillomavirus (HPV).

6. The antibody or the antigen-binding fragment of claim 1 wherein the antibody is polyclonal or monoclonal.

7. The antibody or the antigen-binding fragment of claim 1 wherein the antibody is humanized.

8. A composition for inhibiting infection of a mammalian cell by a papillomavirus, the composition comprises an isolated antibody, or antigen-binding fragment thereof, that binds specifically to residues 36-49 of the papillomavirus (PV) minor capsid L2.

9. The antibody or the antigen-binding fragment of claim 8 wherein the PV is bovine papillomavirus (BPV).

10. The antibody or the antigen-binding fragment of claim 8 wherein the PV is bovine papillomavirus 1 (BPV-1) and wherein the residues 36-49 have a peptide sequence of SEQ ID NO:1.

11. The antibody or the antigen-binding fragment of claim 8 wherein the PV is human papillomavirus (HPV).

12. The composition of claim 8 having a pharmaceutically acceptable carrier.

13. The composition of claim 8 wherein the antibody is polyclonal or monoclonal.

14. The composition of claim 8 wherein the antibody is humanized.

15. A method for inhibiting infection of a mammalian cell by a papillomavirus, the method comprises providing the mammalian cell with an effective amount of a composition comprising a molecule that binds specifically to residues 36-49 of the papillomaviruses (PV) minor capsid L2.

16. The method of claim 15 wherein the PV is bovine papillomavirus (BPV).

17. The method of claim 15 wherein the PV is bovine papillomavirus-1 (BPV-1) and the residues 36-49 have a peptide sequence of SEQ ID NO:1.

18. The method of claim 17 wherein the PV is human papillomavirus (HPV).

19. The method of claim 17 wherein the molecule is a macromolecule or a small molecule.

20. The method of claim 17 wherein the macromolecule is an antibody, or an antigen-binding fragment thereof.

21. The method of claim 20 wherein the antibody is polyclonal or monoclonal.

22. The method of claim 21 wherein the antibody is humanized.

23. A composition for generating an antibody that binds specifically to residues 36-49 of a papillomaviruses (PV) minor capsid L2, the composition comprising a peptide comprising residues 36-49 of a papillomaviruses (PV) minor capsid L2.

24. The composition of claim 23 wherein the peptide is conjugated to keyhole-limpet hemocyannine (KLH) at the N-terminal via a cysteine moeity.

25. An isolated antibody, or antigen-binding fragment thereof, that binds specifically to residues 36-49 (SEQ ID NO:1) of bovine papillomavirus-1 (BPV-1) minor capsid L2.

26. The antibody or the antigen-binding fragment of claim 25 wherein the antibody is capable of inhibiting infection of a mammalian cell by bovine papillomavirus-1 (BPV-1) or human papillomavirus 16 (HPV-16).
